(19) United States
(12) Patent Application Publication
(10) Pub. No.: US 2016/0106344 A1
(43) Pub. Date: Apr. 21, 2016

(54) METHODS AND SYSTEMS FOR DETECTING MOVEMENT DISORDER

(71) Applicant: Milad Nazari, Van Nuys, CA (US)

(72) Inventor: Milad Nazari, Van Nuys, CA (US)

(21) Appl. No.: 14/883,534

(22) Filed: Oct. 14, 2015

Related U.S. Application Data
(60) Provisional application No. 62/122,197, filed on Oct. 14, 2014.
(60) Provisional application No. 62/176,019, filed on Feb. 6, 2015.

(51) Int. Cl. A61B 5/11; A61B 5/0488; A61B 5/0476; A61B 5/00; A61B 5/0402

(52) U.S. Cl. CPC A61B 5/11; A61B 5/7257; A61B 5/0402; A61B 5/0476; A61B 5/0488; A61B 5/6801; A61B 5/0006; A61B 5/7264

(57) ABSTRACT

A platform for diagnosing movement disorder is disclosed. The platform comprises at least one inertia-sensing unit that comprises at least one accelerometer, wherein the inertia-sensing unit measures a user's movement; a power unit that provides power at least to the inertia-sensing unit; a processing unit that performs signal processing on the user's movement and quantifies the movement into a data; a diagnosing unit that analyzes the data to diagnose whether a movement disorder occurs by comparing the data to a database, where the database includes at least information about the user's movement characteristic; and a communication unit that establishes a communication with a computer, a mobile device or a computer server.

### CROSS-REFERENCE TO RELATED PATENT APPLICATIONS

Embodiments of the present invention relate to U.S. Provisional Application Ser. No. 62/122,197, filed Oct. 14, 2014, entitled “Restless Leg Syndrome Aid (RLSA) a device for diagnosing restless leg syndrome” and U.S. Provisional Application Ser. No. 62/176,019, filed Feb. 6, 2015. entitled “Movement disorder diagnosing platform Software Interface Identifies, distinguishes and categorize different Limb and Body. Motions”, the contents of which are incorporated by reference herein in their entirety and which is basis for a claim of priority.

### FIELD OF THE DISCLOSURE

The present disclosure generally relates to systems and methods for diagnosing movement disorder(s). More specifically, the present disclosure discloses a platform that is capable of converting the movements of a user into signals and analyzing the signals to determine whether movement disorder(s) occurred.

### BACKGROUND OF THE INVENTION

Movement disorders are neurological conditions that affect the speed, fluency, quality, and ease of movement. Abnormal fluency or speed of movement (called dyskinesia) may involve excessive or involuntary movement (hyperkinesia) or slowed or absent voluntary movement (hyperkinesa).

Movement disorders generally include the following conditions: Ataxia (lack of coordination, often producing jerky movements), Dystonia (causes involuntary movement and prolonged muscle contraction), Huntington's disease (also called chronic progressive chorea), Multiple system atrophies (e.g., Shy-Drager syndrome), Myocionus (rapid, brief, irregular movement), Parkinson's disease, Progressive supranuclear palsy (rare disorder that affects purposeful movement), Restless legs syndrome (RLS) and reflex sympathetic dystrophy/periodic limb movement disorder (RSD/PLMD), Tics (involuntary muscle contractions), Tourette's syndrome, Tremor (e.g., essential tremor, resting tremor) and Wilson disease (inherited disorder that causes neurological and psychiatric symptoms and liver disease).

For example, Restless legs syndrome (RLS) is one of most common movement disorders characterized by throbbing, pulling, creeping, or other unpleasant sensations in the legs and an uncontrollable and sometimes overwhelming, urge to move them. Symptoms occur primarily at night when a person is relaying or at rest and can increase in severity during the night. Moving the legs relieves the discomfort. Often called paresthesia (abnormal sensations) or dysesthesias (unpleasant abnormal sensations), the sensations range in severity from uncomfortable to irritating to painful.

The most distinctive or unusual aspect of the condition is that lying down and trying to relax activates the symptoms. Most people with RLS have difficulty falling asleep and staying asleep. Left untreated, the condition causes exhaustion and daytime fatigue. Many people with RLS report that their job, personal relations, and activities of daily living are strongly affected as a result of their sleep deprivation. They are often unable to concentrate, have impaired memory, or fail to accomplish daily tasks. It also can make traveling difficult and can cause depression,

According to National Institute of Health website, as many as 10 percent of the U.S. population may have RLS. Several studies have shown that moderate to severe RLS affects approximately 2-3 percent of adults (more than 5 million individuals). An additional 5 percent appears to be affected by a milder form. Childhood RLS is estimated to affect almost 1 million school-age children, with one-third having moderate to severe symptoms. Some people with RLS will not seek medical attention, believing that they will not be taken seriously, that their symptoms are too mild, or that their condition is not treatable. Some physicians wrongly attribute the symptoms to nervousness, insomnia, stress, arthritis, muscle cramps, or aging. RLS occurs in both men and women, although the incidence is about twice as high in women. It may begin at any age. Many individuals who are severely affected are middle-aged or older, and the symptoms typically become more frequent and last longer with age.

RLS occurs in both men and women, although the incidence is about twice as high in women. It may begin at any age. Many individuals who are severely affected are middle-aged or older, and the symptoms typically become more frequent and last longer with age.

More than 80 percent of people with RLS also experience a more common condition known as periodic limb movement of sleep (PLMS). PLMS is characterized by involuntary leg twitching or jerking movements during sleep that typically occur every 15 to 40 seconds, sometimes throughout the night. The symptoms cause repeated awakening and severely disrupted sleep. Although many individuals with RLS also develop PLMS, most people with PLMS do not experience RLS. People who have PLMS and do not have RLS or another cause for the PLMS may be diagnosed with periodic limb movement disorder (PLMD). PLMD may be a variant of RLS and thus respond to similar treatments.

Currently, there is no specific test for RLS. The four basic and conventional criteria for diagnosing the RLS are: 1) Symptoms that are worse at night and are absent or negligible in the morning: 2) A strong and often overwhelming need or urge to move the affected limb(s), often associated with paresthesia or dysesthesias: 3) Sensory symptoms that are triggered by rest, relaxation, or sleep; and/or 4) Sensory symptoms that are relieved with movement and the relief persists as long as the movement continues.

Physicians usually focus largely on the individual's descriptions of symptoms, their triggers and relieving factors, as well as the presence or absence of symptoms throughout the day. Sometimes a neurological and physical exam is administered to the patients along with the information from the individual's medical and family history and list of current medications to determine whether an individual has RLS. Individuals may be asked about frequency, duration, and intensity of symptoms as well as their tendency toward daytime sleep patterns and sleepiness, disturbance of sleep, or daytime function.

Laboratory tests may be performed to rule out other conditions. Blood tests can identify iron and vitamin deficiencies as well as other medical disorders associated with RLS. In some cases, sleep studies such as polysomnography (a test that records the individual's brain waves, heartbeat, breathing, and leg movements during an entire night) may identify the presence of other causes of sleep disruption (e.g., sleep apnea), which may impact management of the disorder.

Diagnosing RLS in children may be especially hard, since it may be hard for a child to describe where it hurts, when and how often the symptoms occur, and how long symptoms last. Pediatric RLS can sometimes be misdiagnosed as “growing pains” or attention deficit disorder.

Once RLS has been identified, there are many ways to treat RLS. For instances, RLS can be treated, with care directed toward relieving symptoms. Moving the affected limb(s) may provide temporary relief. Sometimes RLS symptoms can be controlled by finding and treating an associated medical condition, such as peripheral neuropathy or diabetes.

Certain lifestyle changes and activities that may reduce symptoms in persons with mild to moderate symptoms include decreased use of caffeine, alcohol, and tobacco; supplements to correct deficiencies in iron, folate, and magnesium; changing or maintaining a regular sleep pattern; a program of moderate exercise; and massaging the legs, taking a hot bath, or using a heating pad or ice pack. A trial of iron supplements is recommended only for individuals with low iron levels. Although many people find some relief with such measures, rarely do these efforts completely eliminate symptoms.

Medications are usually helpful but no single medication effectively manages RLS for all individuals. Trials of different drugs may be necessary. In addition, medications taken regularly may lose their effect over time making it necessary to change medications periodically.

Common drugs prescribed to treat RLS include:

Dopaminergic agents (drugs that increase dopamine), largely used to treat Parkinson's disease, have been shown to reduce symptoms of RLS and PLMS when they are taken at bedtime and are considered the initial treatment of choice. The U.S. Food and Drug Administration (FDA) has approved ropinirole, pramipexole, and rotigotine to treat moderate to severe RLS. Both drugs are generally well tolerated but can cause nausea, dizziness, or other side effects. Good short-term results of treatment with levodopa plus carbidopa have been reported.

Although dopamine-related medications are effective in managing RLS, long-term use can lead to worsening of the symptoms in many individuals. This apparent progressive worsening is, referred to as “augmentation.” With chronic use, a person may begin to experience symptoms earlier in the evening than in the afternoon until finally the symptoms are present around the clock. The initial evening or bedtime dose becomes less effective, the symptoms at night become more intense and symptoms begin to affect the arms or trunk. Fortunately, this apparent progression is reversible by removing the person from all dopamine-related medications. Another important adverse effect of dopamine medications that occurs in some people is the development of impulsive or obsessive behaviors such as obsessive gambling or shopping. Should they occur these behaviors can be reversed by stopping the medication.

The FDA has approved gabapentin enacarbil, which metabolizes in the body to become gabapentin, for the treatment of moderate to severe RLS.

Other medications may be prescribed “off-label” (not specifically designed to treat RLS) to relieve some of the symptoms of the disorder.

Benzodiazepines can help individuals who have mild or intermittent symptoms obtain a more restful sleep. However, even if taken only at bedtime they can sometimes cause daytime sleepiness. Benzodiazepines such as clonazepam and diazepam are generally prescribed to treat anxiety, muscle spasms, and insomnia. Because these drugs also may induce or aggravate sleep apnea in some cases, they should not be used in people with this condition.

Opioids such as codeine, propoxyphene, or oxycodone may be prescribed at night to diminish pain and help to relax individuals with more severe symptoms. Side effects include dizziness, nausea exacerbation of sleep apnea, and the risk of addiction.

Anticonvulsants such as gabapentin and pregabalin can decrease the sensory disturbances such as creeping and crawling sensations and nerve pain. Dizziness, fatigue, and sleepiness are among the possible side effects.

The Relaxis pad, which the person can place at the site of discomfort when in bed and provides 30 minutes of vibrations (counterstimulation) that ramp off after 30 minutes, has been approved by the FDA for individuals with RLS.

Another device generally used for the symptomatic relief of chronic pain is a Transcutaneous Electrical Nerve Stimulation (TENS) unit, as described in U.S. Published Patent Application No. 20130158627. A TENS unit may often be used for restless or seized muscle issues, but current TENS technology is greatly limited in its overall effectiveness because the stimulation has a habituation effect, wherein a decrease in sensory perception of a stimulus occurs after prolonged presentation of the stimulus. Thus, the result is the patient is forced to constantly turn up the stimulus to feel the same effect. The improved TENS unit of the 20130158627 published application learns the manner and frequency of the manual adjustment of the user over time to customize parameters to assist with habituation. However the unit still requires manual adjustments from the user, while setting the parameters and does not utilize any physiological data to determine the effect the stimulation is having on the user. There have been many cases of user error with TENS technology that has resulted in injury to the user because some users turn the stimulation higher than needed for the portion of the body being treated and muscle or tissue injury can occur. Thus, an automatic TENS (ATENS) unit that could detect the physiological effects the stimulation the unit is having on the treated tissue, and have diagnostic capabilities which feedback to the unit in a closed loop or automated way whether the stimulus should be raised or lowered or is having an effect at all could greatly advance the field of either treating the chronic pain currently TENS technologies, or additionally serve as a counter-stimulation mechanism for various involuntary movement disorders.

Since the current diagnosis is qualitative, there is no numeric understanding of the symptoms, causing confusion amongst doctors and patients on how bad patients' conditions are. In the meantime, some patients are not diagnosed properly (they get diagnosed for wrong medical conditions or disorders) or are under-diagnosed for their disorders. Finally, there are times that doctors have difficulty in prescribing the right amount of medication since there is no numeric representation of a RLS patient's severity of symptoms.

Accordingly, there exists a need for improved methods and systems for detecting movement disorder. There also exists a need for improved systems and methods that allow quantitative analysis for symptoms of movement disorder. There also exists a need for improved systems and methods that automatically provide relief to patients' muscle pain or interfere with patients' muscles active potentials once movement disorder is detected.

### SUMMARY OF THE INVENTION

One of present disclosure's goals is to promote human health with the advanced diagnostic tools which are available to everyone at this day and age, and combine them with innovative cutting-edge art of technology, to help correct the correlation of human body in such cases as of movement disorders. The present disclosure, with its precise supplementary tools, such as the added electromyography (EMG), electroencephalogram (EEG) and/or electrocardiogram (ECG), will be a great pioneer in this radical technology era. All collected necessary and efficient data, for our reference and base, will then be available to us at our fingertip, worldwide, anywhere, anytime.

Accordingly, one objective of the present disclosure is to disclose a system and method capable of correctly identify movement disorder(s) on patients.

Another objective of the present disclosure is to disclose a system and method that accurately diagnoses movement disorders such as restless leg syndrome by finding the bio print of a person's normal movement pattern and detecting movements that are inconsistent with the bio print of the person.

Another objective of the present disclosure is to disclose a system and method that is capable of providing the most effective procedure to relieve patients' muscle pain or interfere with patients' muscles active potentials such as using the right amount of Transcutaneous Electrical Nerve Stimulation (TENS) amplitude and pulse once movement disorder is detected.

In accordance with one aspect of at least one embodiment of the present disclosure, a platform for diagnosing movement disorder is disclosed. The platform comprises: 1) at least one inertia-sensing unit that comprises at least one accelerometer and at least one gyroscope, wherein the inertia-sensing unit performs three-dimensional measurement of a user's movement; 2) a power unit that provides power to the inertia-sensing unit; 3) a processing unit that performs signal processing on the three-dimensional measurement of the user's movement and quantifies the movement into a signal; 4) a diagnosing unit that analyzes the signal to diagnose whether a movement disorder occurs by comparing the signal to signals in a database, where each of the signals in the database is associated with a normal or an abnormal movement of the user or a typical person; and 5) a communication unit that establishes communication with a computer, a mobile device or a computer server.

In one embodiment of the present disclosure, the disclosed system is part of an Internet of Things (IOT) package; hence, the package includes one or more electronic wearables, one or more application software, and one or more servers, and scientific algorithms that are used for diagnosis purposes. For example, and not by way of limitation, DFT (discrete Fourier transform), FFT (fast Fourier transform) or other variation of Fourier transform that calculate frequency” are used to calculate and quantify a user's movement.

In yet another embodiment of the present disclosure, the disclosed system further comprises an automotive TENS unit that uses Artificial Intelligence and machine learning to learn the patterns of the movement disorder. Then the Automotive TENS apparatuses interfere with the muscle's active potentials in order to automatically relieve the pain on the muscles and to weaken the active potential signal; thus, reducing the movements associating with movement disorder.

The present disclosure uses real-time data generated by inertia sensors. The disclosed platform identifies distinguishes and categorizes different limb and body motions including but not limited to idle periodic motions, non-idle periodic motions, locomotive periodic motions and non-periodic motions from each other. The disclosed system further interacts with the user and reports its findings through a user-friendly interface. The system's algorithms use the dot product of functions to transform data from a span of a ‘k’ dimensional vector space into a span of ‘n’ dimensional vector space. Using a combined set of vectorial and scalar analyzed ‘movement characteristics’ components and scalar, the application distinguishes idle periodic motions including but not limited to the Restless Leg Syndrome limb's motion characteristics from non-idle periodic motions, locomotive periodic motions and non-periodic motions. As part of the process, the system is able to distinguish those frequencies of disorders including but not limited to the Restless Leg Syndrome limbs' motion from other type of movements such as driving, walking, running and etc.

These and other features, aspects and advantages of the present invention will become better understood with reference to the following drawings, description and claims.

### BRIEF DESCRIPTION OF THE DRAWINGS

FIG. 1 is a schematic diagram according to an exemplary embodiment of the present invention.

FIG. 2 is a schematic diagram according to an exemplary embodiment of the present Invention.

FIG. 3 is a schematic diagram according to an exemplary embodiment of the present invention.

FIG. 4A is an example of a vertical acceleration (Y Axis) FFT analysis for walking according to an exemplary embodiment of the present invention.

FIG. 4B is an example of vertical acceleration (Y Axis) FFT analysis for RLS according to an exemplary embodiment of the present invention.

FIG. 5 is a schematic diagram according to an exemplary embodiment of the present invention.

FIG. 6 is a schematic diagram according to an exemplary embodiment of the present invention.

FIG. 7 is a schematic diagram according to an exemplary embodiment of the present invention.

FIG. 8 is a schematic diagram according to an exemplary embodiment of the present invention.

FIG. 9 is a schematic diagram according to an exemplary embodiment of the present invention.

FIG. 10 is a schematic diagram according to an exemplary embodiment of the present invention.

FIG. 11 is a schematic diagram according to an exemplary embodiment of the present invention.

FIG. 12 is a schematic diagram according to an exemplary embodiment of the present invention.

FIG. 13 is a schematic diagram according to an exemplary embodiment of the present invention.

FIG. 14 is a schematic diagram according, to an exemplary embodiment of the present invention.

### DETAILED DESCRIPTION OF THE PREFERRED EMBODIMENT(S) AND INVENTION

The following description is not to be taken in a limiting sense, but is made merely for the purpose of illustrating the general principles of the disclosure, since the scope of the disclosure is best defined by the following claims. Various inventive features are described below that can each be used independently of one another or in combination with other features.

While the illustrations below may focus on restless leg syndrome, please note that neither the application/usage of the disclosed systems and methods nor the scope of this invention is limited to usage on restless leg syndrome. With little or no modification, the same invention is usable in the diagnoses of a variety of movement disorders, including but not limited to Ataxia, Dystonia, Huntington's disease. Multiple system atrophies. Myoclonus, Parkinson's disease. Progressive supranuclear palsy, and reflex sympathetic dystrophy/periodic limb movement disorder. Tics, Tourette's syndrome, Tremor and Wilson disease. In addition, all the units/software/components used by platform of the present disclosure communicate with each either wirelessly or physically. The units/software/components include the inertia-sensing unit 4, communication unit 14, microcontroller 38, user interface, processing unit 6, diagnosing unit, database 12, electromyography unit 20, electrocardiogram unit 24, electroencephalogram unit 22, blood pressure sensor 36 and the actuator 40.

For purposes of the present disclosure, the term “movement disorder” shall refer to any neurological condition that affects the speed, fluency, quality, and ease of a person's movement. The term “wearable device” shall include assembly and/or mechanism, in any form and type that can be attached to arty part of a person. The term “inertial sensor” or “motion sensor” shall refer to any sensor that senses inertia. For example, and not by way of limitation, inertial sensor includes accelerometers, gyroscopes and piezoelectric sensor. The term “biometric” or “bio print” refers to metrics related to human characteristics which may identify unique biological characteristics of a living organism. The term “idle periodic movement” refers to the movement in which one part of the body moves periodically while the entire body frame is fixed at a position. The term “locomotive periodic movement” refers to the movement in which one part of the body moves periodically while the entire body frame is moving in one axis. The term “non-idle periodic movement” refers to the movement in which one part of the body moves periodically while the entire body frame is moving two axis. For instance when a person climbs the stairs he is moving both in x, and y direction. The term “non-periodic movement” refers to the movement in which a part of body moves randomly.

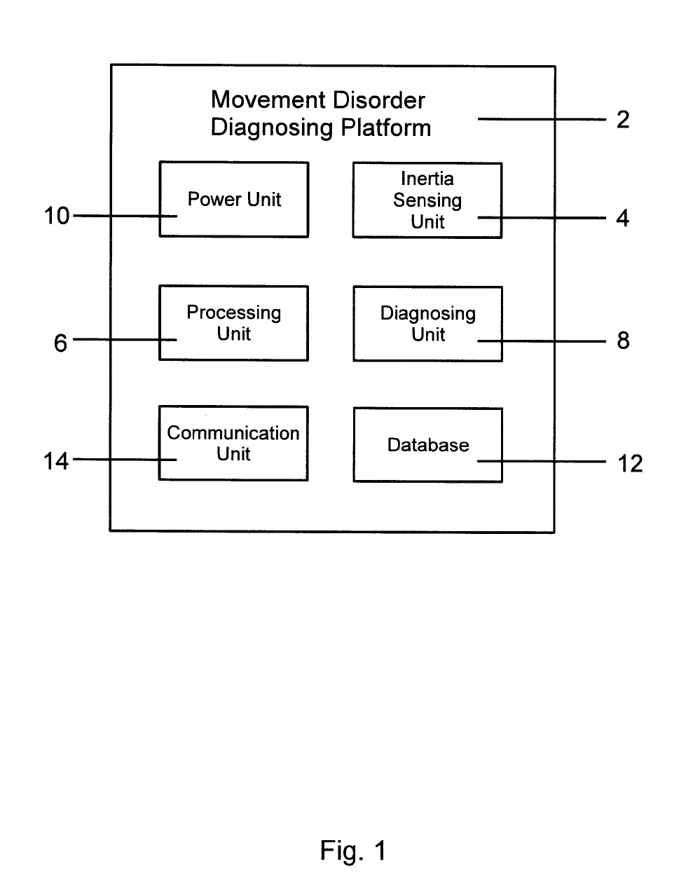

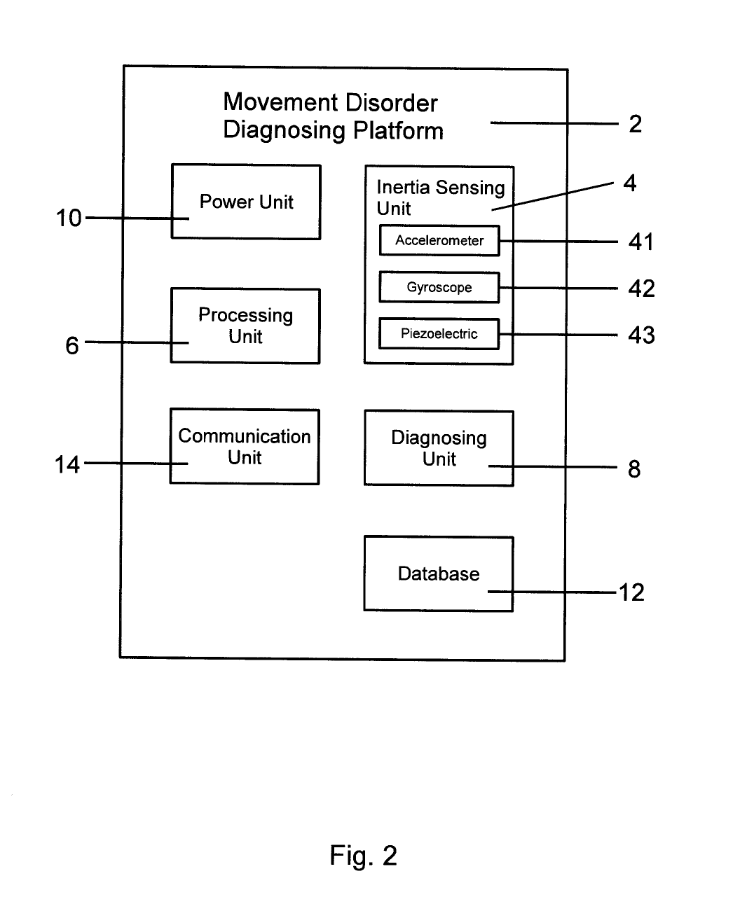

Broadly, embodiments of the present disclosure generally provide a platform for diagnosing, movement disorder(s). As shown by FIGS. 1 and 2, in one embodiment of the present, disclosure, the disclosed platform 2 comprises: 1) at least one inertia-sensing unit 4 that comprises at least one accelerometer 41 and optionally at least one gyroscope 42 and/or at least one piezoelectric sensor 43, wherein the inertia-sensing unit 4 performs measurement of a user's movement inertia; 2) a power unit 10 that provides power to the inertia-sensing unit and optionally to other unit(s) in the platform (e.g., communication unit 14, microcontroller 38 user interface etc.) 3) a processing unit 6 that performs signal processing on the measurement of the user's movement and quantifies the movement into a data (which may also be referred to as “signal” throughout the specification) 4) diagnosing unit 8 that analyzes the data to diagnose whether a movement disorder occurs by comparing the data to a database 12, where the database 12 includes at least information about the user's movement characteristic (e.g., the database 12 may prerecords information about which particular movement is correspond to idle periodic movement, non-idle periodic movement, locomotive periodic movement and non-periodic movement of the user): and 5) a communication unit 14 that establishes communication with a computer, a mobile device or a computer server, or establish communication between different components/units within the disclosed platform 2.

In one embodiment of the present disclosure, if the inertia-sensing unit 4 uses at least one gyroscope 42, then the inertia-sensing unit 4 is capable of additionally measuring the user's movement orientation, thereby providing three-dimensional measurement of the user's movement inertia. The combination of the user's movement orientation and the movement measured by the accelerometer provide higher accuracy for the platform's movement disorder diagnosis.

There are two levels to the diagnosis process: First part is to perform calculations on the signals (Signal Processing). Second part is to have the processed signals that could be referred to as “the pre diagnosed data” compared to the diagnosis characteristics of the database 12. Thus, the diagnosis unit 8 can be a hybrid concept that could either be partially implemented by hardware or partially by the software, or that it could be implemented using software and server only, or it could be implemented using hardware, software and server. For instance: The processing unit 6 of the platform 2 could either send the raw data to a computer, a mobile device or a computer server via the communicational unit or that it could perform calculations on the signals (Signal Processing) and send pre diagnosed data to a computer, a mobile device or a computer server. In result having diagnosing unit 8 as part of the device is optional.

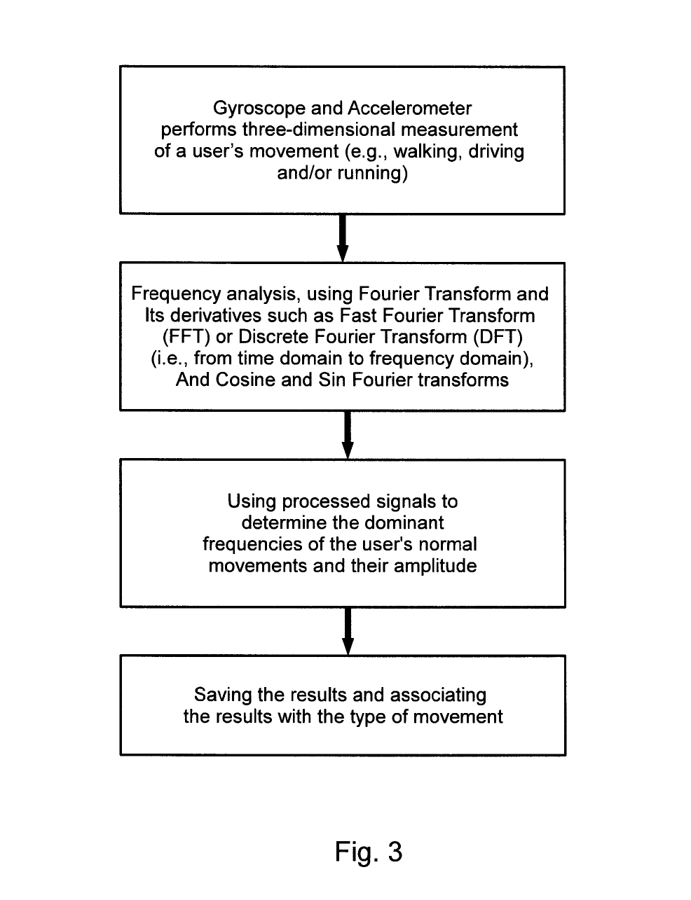

In one embodiment of the present disclosure, as shown by FIG. 3, the accelerometer 41 and the gyroscope 42 of inertia-sensing unit 4 performs three-dimensional measurement of a user's movement (e.g., while the user is walking, driving; and/or running). For example, and not by way of limitation, the three-dimensional measurement includes measuring specific force and angular rate of the user's movement. Then the processing unit 6 receives the three-dimensional measurement of a user's movement and performs signal processing on the user's movement, such as frequency analysis, using Fourier Transform and its derivatives such as Fast Fourier Transform (FFT) or Discrete Fourier Transform (DFT) (i.e., from time domain to frequency domain), and Cosine and Sin Fourier transforms. For instance, for any given vector in x,y,z coordinate, the projection of vector V can be calculated and converted into the standard bases function used for Fourier Transform {e-i2πωx, ωεR} or any other variations of the Fourier transform such as Cosine and sin Fourier transforms that calculate the frequency of a signal. This way the frequencies of different signals that compose the main signal can be discovered. The signal processing can be performed by a hardware, a software, or a combination or software and hardware (e.g., hybrid system).

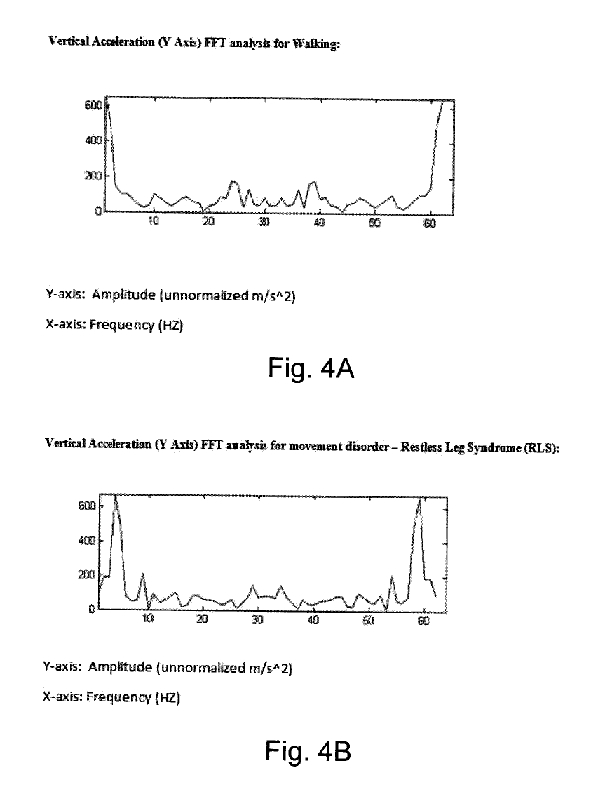

FIG. 4A shows an example of a vertical acceleration (Y Axis) FFT analysis for walking and FIG. 4B shows an example of vertical acceleration (Y Axis) FFT analysis for RLS. One of the biggest advantages of performing signal processing on a movement and turn it to a signal is to allow quantitative analysis of the movement. The signal can also serve as a biometric or bio print for the user because everyone moves differently (e.g., different rhythm tempo and speed).

Frequency analysis, using Fourier Transform and its derivatives such as Fast Fourier Transform (FFT) or Discrete Fourier Transform (DFT) (i.e., from time domain to frequency domain), and Cosine and sin Fourier transforms can be the most reliable way of distinguishing periodic movements such as walking, and RLS from each other.

The following example and description is not to be taken in a limiting sense, but is made merely for the purpose of illustrating the general principles of the disclosure, since the scope of the disclosure is best defined by the following claims. As FIG. 4A illustrates, the FFT of walking provides with the frequencies that compose a walking signal in time domain. Meanwhile, FIGS. 4A and 4B provides a comparison between walking's and RLS movement's FFT. In this example we once calculate the FFT of the sample walking data and calculate the FFT of every receiving 64 samples of the microcontroller (64×10 ms=0.64 s). By comparing the frequency ranges of these two FFT, we would be able to see whether a movement could be categorized as RLS or not.

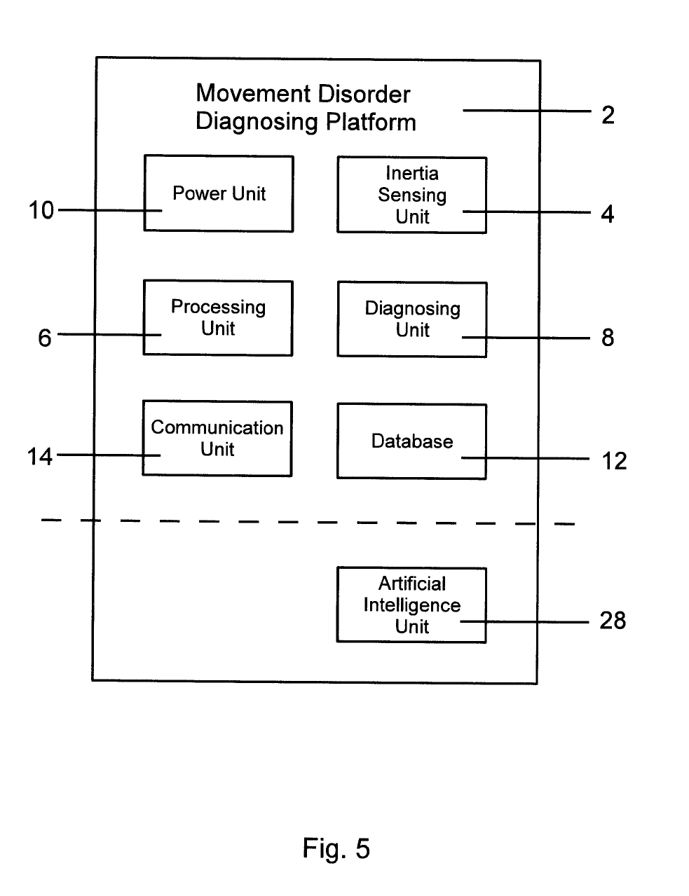

In one embodiment of the present disclosure as shown by FIG. 5, the platform 2 further comprises an artificial intelligent unit 28, which identifies and records the signal with its associated movement and constantly updates the signals in the database 12. Signals in the database 12 may be categorized into idle periodic movement, non-idle periodic movement, locomotive periodic movement and non-periodic movement, wherein the idle periodic movement is usually associated with movement disorder. The signal is then used by the artificial intelligent unit 28 and/or diagnosing unit 8 to determine the dominant frequencies of the user's normal movements and their amplitudes. Then the artificial intelligent unit 28 associating each signal with a type of user's movement (eg., walking, running, jumping etc.). The artificial intelligence unit 28 is an intelligence exhibited by machines, software, or a combination of both. An example of artificial intelligence is the machine learning, which is the study of computer algorithms that improve automatically through experience and has been central to artificial intelligence research since the field's inception. Hybrid Artificial Intelligence may also be used, which is a concept comprised of software algorithms or hardware designs to learn from patterns or to make appropriate predictions about future patterns of the movement disorder's patterns.

For example and not by way of limitation, the artificial intelligent unit 28 is a software algorithm or a hardware or combination of both that learns from patient's symptoms including but not limited to the frequency of the movement disorder, the periodicity of the movement disorder, or the change in movement characteristics such as speed, velocity, acceleration, limb's orientation with the respect to the reference frame.

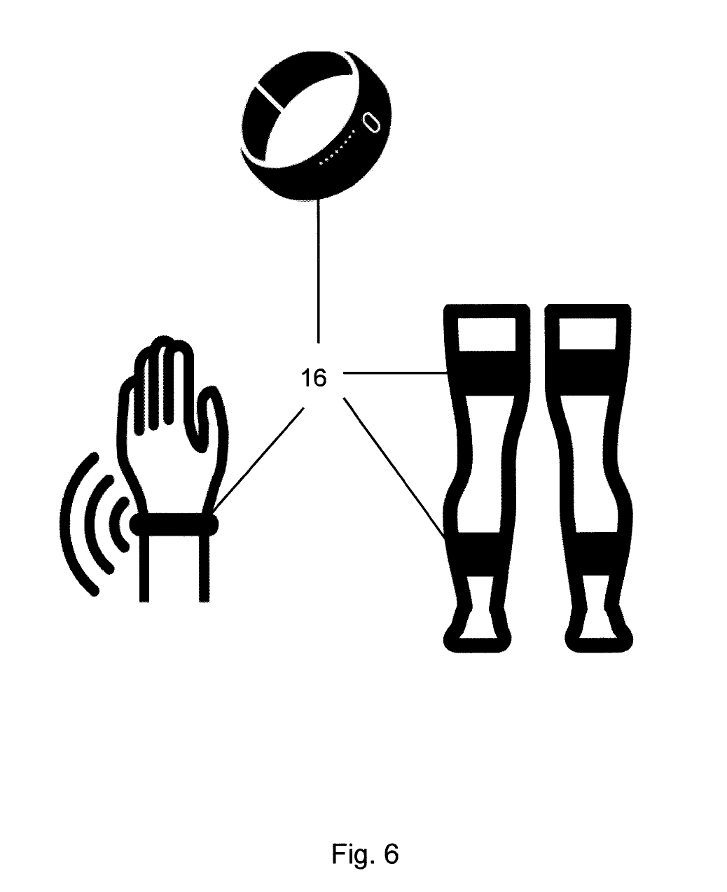

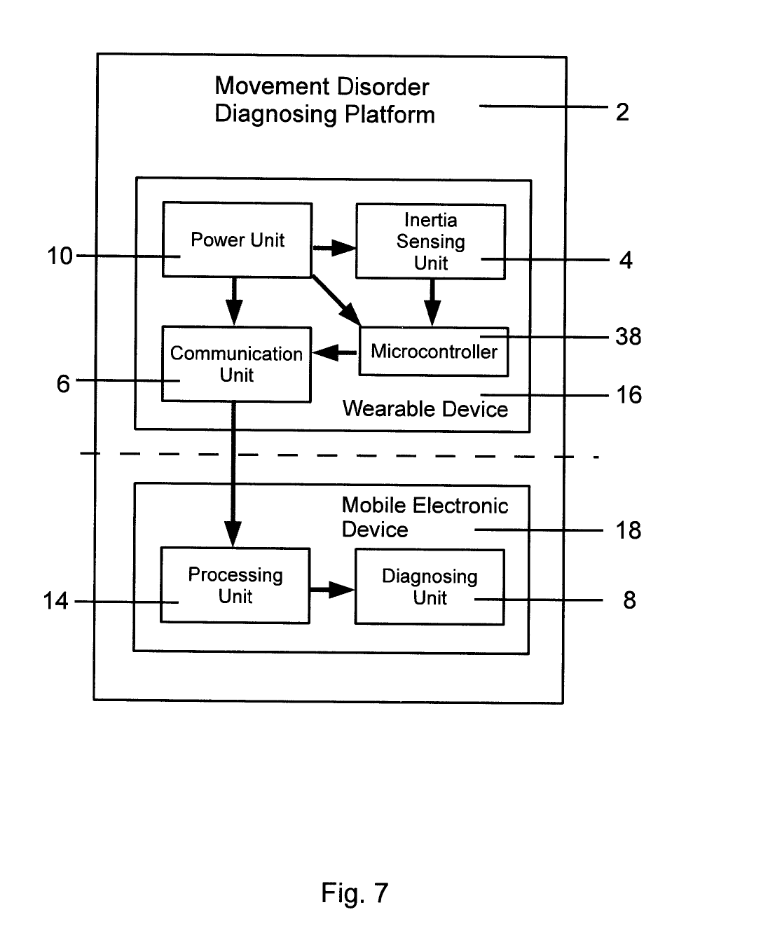

In yet another embodiment of the present disclosure, various units on the disclosed platform can be installed on a wearable device 16 such as stretchable fabric which can be worn by the user on its hand or leg as shown by FIG. 6. For example, as shown by FIG. 7, the power unit 10, the inertia-sensing unit 4 and the communication unit 14 are on a wearable device whereas the processing unit 6 and the diagnosing unit 8 are on an electronic device 18 (i.e., as a software on a computer or mobile application on a mobile device). The processing unit 6 and the diagnosing unit 8 can also be an algorithm, software, hardware or a combination of software and hardware (e.g., hybrid system). The wearable device 16 may further comprise a microcontroller 38 for receiving movement information from the inertia-sensing unit 4 and is powered by the power unit 10 as well. Then the microcontroller can be a processor that passes out the movement information to the communication unit 14. The communication unit 14 allows the wearable device 16 to communicate with the electronic device 18 through wire or wirelessly (e.g., using typical wireless modules). Please note that the example above shows only one embodiment of the present disclosure is not by any means limiting the scope of the present disclosure. Various units of the platform (including the ones discussed below) can also be placed on the wearable device instead of the electronic device and vice versa. For instance, the processing unit 6 and the diagnosing unit 8 can be a separate piece of hardware placed on the wearable device 16, thereby eliminating the requirement of having an additional electronic device 18. As such, the processing unit 6, the diagnosing unit 8 and/or the artificial intelligent unit 28 can be algorithm, software, hardware or a combination of them. The communication unit 14 also has the capability to establish communications between different components/units within the disclosed platform 2.

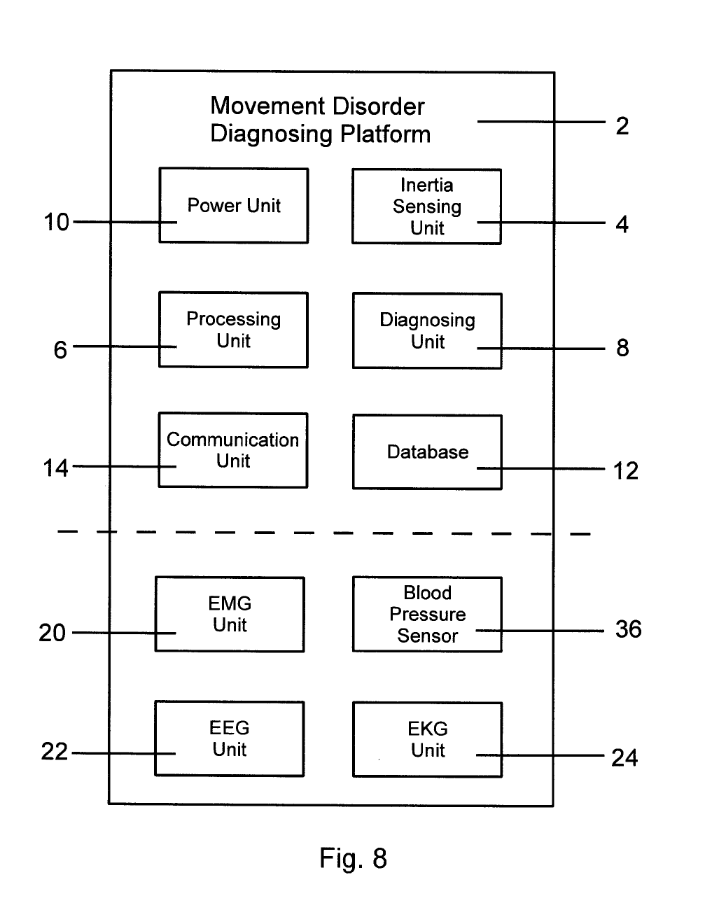

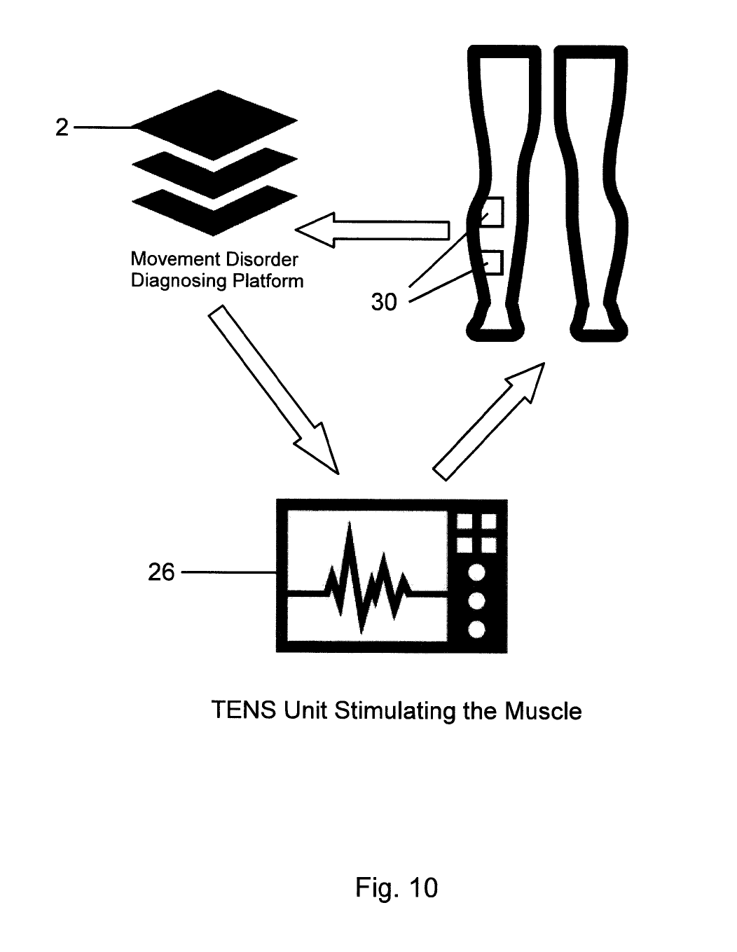

In yet another embodiment of the present disclosure, in order to enhance the accuracy of the platform's 2 diagnosis, platform 2 further comprises an electromyography (EMG) unit 20, an electrocardiogram (EKG or ECG) unit 24 an electroencephalogram (EEG) unit 22, a blood pressure sensor 36 or a combination thereof as shown by FIG. 8. The EMO unit 20 detects and measures change in active potentials of the user's muscle and may independently diagnose whether a particular movement of the user is normal. For example, the EMG unit 20 detects muscles active potential signals via the electrodes (see electrodes 30 in FIG. 10) that are connected to the skin's surface (neurological signal). In yet another embodiment, the artificial intelligent unit 28 uses the EMG unit 22 to probe whether the electrical current applied to the user is effective and adjusts the EMG unit's 22 electrical current level accordingly to provide most effective level of electrical current to the user. The EEG unit 22 probes the user's brain activity for determining the effect of brain activity on movement disorder. The ECG unit probes the user's heart activity for determining the effect of heart activity on movement disorder. The blood pressure sensor 36 probes the user's heart activity for determining the effect of blood pressure on movement disorder. The diagnosing or probing result(s) performed by the EMG unit 20, the EKG unit 24, the EEG unit 22 and/or the blood pressure sensor 36 is then correlated with the diagnosing unit's 8 diagnosis to enhance the platform's 2 accuracy for diagnosing movement disorder. These additional unit(s) and sensor(s) can be part of the wearable device or as an independent device that communicates with each other through wire or wirelessly. For example, they can communicate to diagnosing unit 8 and/or artificial intelligence unit 28 on a mobile device or with each other through Bluetooth, area network, Wi-Fi and/or Li-Fi.

With the addition of the EMG unit 20, the diagnosis of movement disorder can be done in two forms: 1) Using Accelerometer and Gyroscope and 2) Using EMG for Diagnosis.

Using Accelerometer and Gyroscope: as previously stated, the FFT of acceleration is calculated and the frequency of the limb's motion for x, y and z access can be known. By looking at the results from the gyroscope 28, the orientation of a persons' limb is discovered as well. Once the two data types are combined, the device would be able to distinguish between the idle periodic movement and the locomotive periodic movement. However, if the person is stationed in a vehicle, such as a car, the acceleration data might show spurious periodic limb movement not associated with movements initiated by the user; thus, this might be mistaken with periodic limb movement. As such, in yet another embodiment of the present disclosure, the disclosed platform is capable of detecting the speed of the user relative to a ground using UPS or any type of positioning system similar to GPS. GPS data, which could be provided from the hardware of the mobile electronic device 18 (e.g., mobile phone) or as an independent unit on the wearable device 16 are used by algorithm of the platform 2 to calculate the speed of the movement as well as the location of the user in order to determine person's frame of movement type. For instance, if the speed is above a certain number and the location of movement is a road the platform 2 will determine that the person is stationed in a car; hence, it will use other algorithms to calculate repetitive movements of the limb.

Using Electromyography (EMG) for Diagnosis: the change in active potentials of the muscles are discovered by the EMG circuit and are sent to the platform; there the magnitude and the periodicity of the signals are processed based on whether the movement matches periodic limb's movement characteristics or not.

Correlating the EMG diagnosis with the acceleromete and gyroscope's data: since the readings from the accelerometer gyroscope and EMG is done in real-time; thus, the algorithm used by the platform 2 will associated EMG's signal activity with the amplitude of the limb movement that the person makes. Thus, enabling to estimate Muscle's EMG activity using accelerometers data or vice versa.

In yet another embodiment the diagnosis platforms, described above, may provide information about the efficacy of treatment with either therapeutics or medical devices or various other treatment regimens, thus allowing for a more customized and effective treatment of each user's involuntary movement disorder.

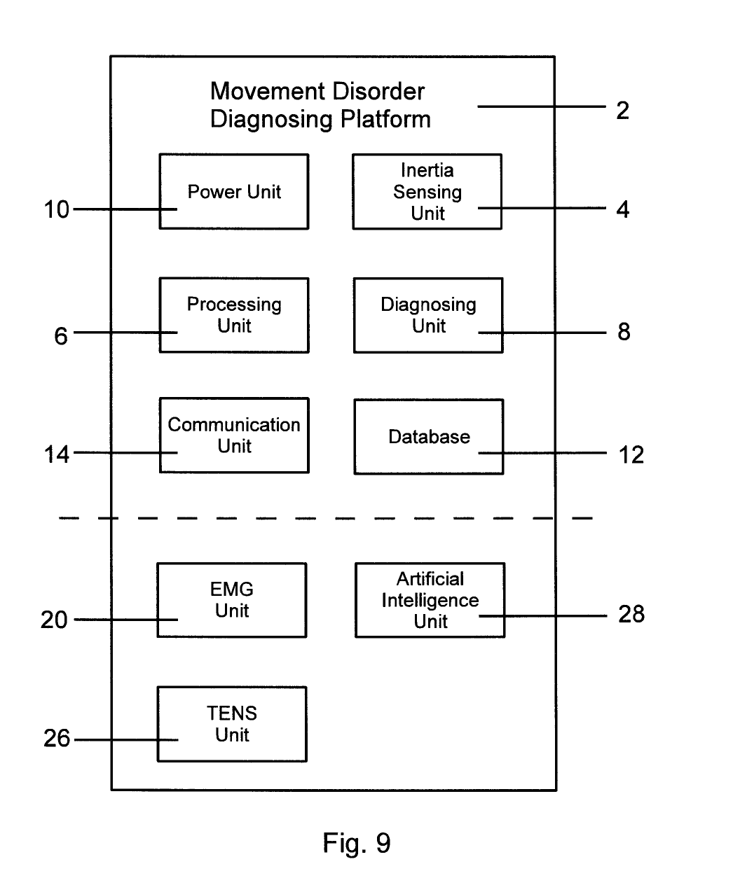

In yet another embodiment of the present disclosure, as shown by FIG. 9, the platform 2 further comprises a unit 26. The TENS unit 26 automatically applies therapeutically effective amount of electrical currents to the user when an abnormal movement is detected to suppress acute and chronic pain caused by the abnormal movement. In addition, the combination of artificial intelligence 28, the EMG unit 20 and the TENS unit 26 allows the platform 2 to become a smart automative transcutaneous electrical nerve stimulator (ATENS). ATENS stimulates the muscle and looks for the feedback and it repeats the process until it finds the right point of stimulation. For example, and not by way of limitation, as shown by FIG. 10, once the platform 2 diagnoses a movement disorder, the platform 2 notifies and activates the TENS unit 26. Then the TENS unit 26 applies certain amount of electrical currents to the user via the electrodes 30. The electrodes 30 may optionally be designed to be automatically adjustable such that the electrodes can move on the user's body to find most effective location/position of the body to administer the treatment (i.e., to apply electrical current). The platform 2 will keep monitoring the movement of user (or the user's muscle) while the electrical currents are delivered and constantly adjusts the amplitude of the electrical currents to optimize the effectiveness of TENS unit 26. For instance, the amplitude of electrical current is increased if the user shows no reaction and decreased if the user shows drastic reaction.

In yet another embodiment of the present disclosure, the disclosed ATENS further comprises an user interface allowing the user to manually adjust the parameters of the diagnosing platform 2 and or its unit(s)component(s) (i.e., the diagnosing unit 28, TENS unit 26 and/or EMG unit 20), thereby assisting the artificial intelligent unit 28 to learn or to better understand the user's symptom pattern; thus allowing the diagnosing platform 2 to apply more accurate amount of stimulation.

A more natural way of dealing movement disorder such as severe RLS symptoms is by using TENS unit 26. A patient may utilize TENS unit 26 to apply electrical stimulation to their limb to reduce their limb sensations. In general, TENS unit 26 applies electrical currents to a particular area of the human body it order to suppress acute and chronic pain. For most common TENS unit 26, electrodes are placed on the skirt within adjacent to, or proximal to, the area of pain. Electrical stimulation is then delivered to the patient through the electrodes, with the electrical stimulation being in the form of low intensity (typically less than 50-60 mA) short duration (typically 50-200 μsec) pulses at frequencies typically between about 10 and 200 Hz.

In addition, the electrical circuit of TENS unit 26 generates stimulation pulses with specified characteristics. The pulse waveform specifications include intensity (mA), duration (μsec) and shape (typically monophasic or biphasic). The pulse pattern specifications include frequency (Hz) and length of the stimulation session (minutes). One or more pairs of electrodes, placed on the patient's skin, transduce the electrical pulses and thereby stimulate underlying nerves. By varying the intensity of the stimulation pulses and, to a lesser degree, the frequency of the stimulation pulses, the clinical benefit of TENS can be optimized.

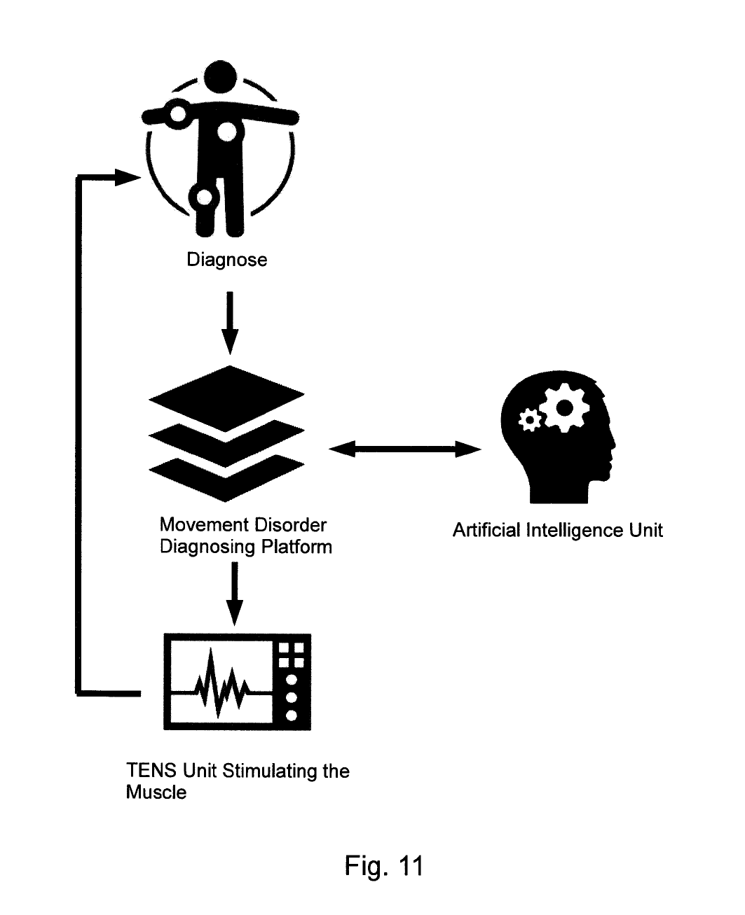

FIG. 11 illustrates another example of using TENS Unit 26 for therapy. An automotive TENS unit 26 is added to the platform and controlled by the artificial intelligence unit 28 and/or diagnosing unit 8. Without user's involvement the platform 2 automatically sets the voltage and required signal amplitude to overcome muscle's habituation. Every time the TENS unit 20 is activated the EMG unit 20 probes muscles' change of active potential signal and when it finds a range of amplitude and voltage in which the muscle is reacting it automatically sets the range for TENS functionality.

At the same time, the artificial intelligence unit 28 records all the data obtained (i.e., amount of voltage to apply in response to a movement disorder) and learns from the user's symptom pattern and uses those to calibrate TENS unit's 20 performance such as the voltage on two sides of the electrodes, the amplitude of the current conducted into the limb's muscle and the duration of this operation.

By collecting the data of the TENS units performance the diagnosis platforms may be further able customize treatments related to the feedback received about the stimulations effects on the targeted tissue or muscle. The resulting customization of stimulus and/or automatic stimulus response can adjust to the natural habituation which occurs with traditional TENS devices, while protecting the user from user error by either under-stimulating or over-stimulating the treated muscle or tissue. Additional advantages may be able to counter-stimulate the current conducted into a muscle during an involuntary muscle movement event, and/or have an effect on the root of the central nervous system which is stimulating the involuntary movement. The embodied counter-stimulation techniques may block or redirect the impulses sent during an involuntary movement event or may even retrain the brain to stop sending the impulses.

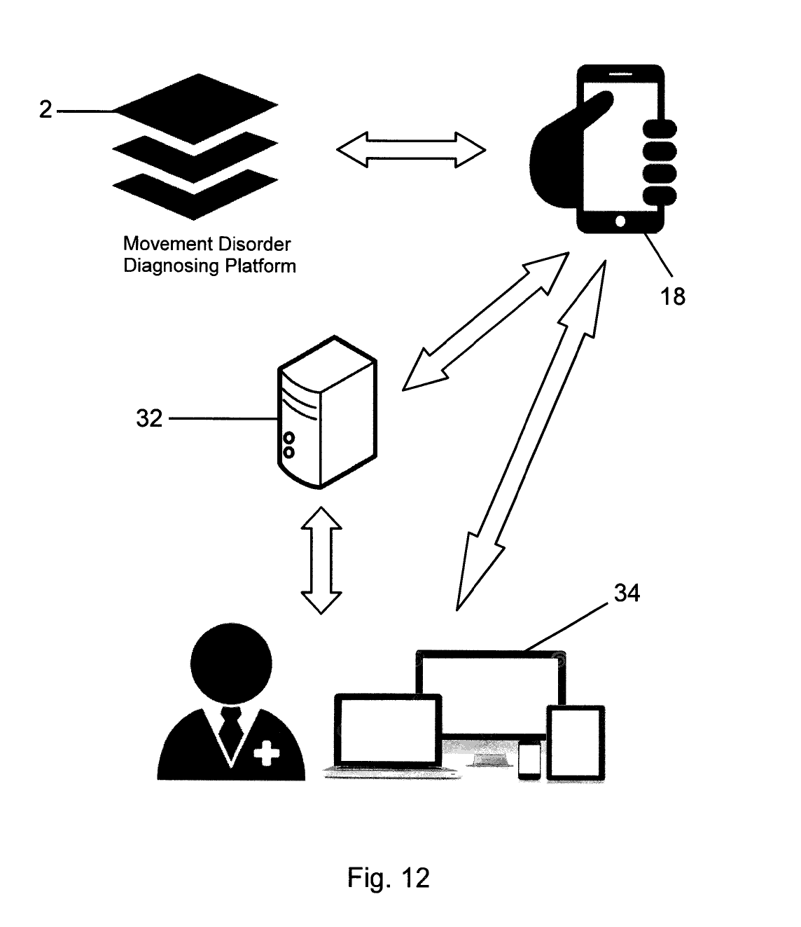

In yet another embodiment or the present disclosure, as shown by FIG. 12 the mobile electronic device 18 of the platform 2 transmits the diagnosing result directly or indirectly to a physician 34 or a medical platform/server 32, and the diagnosing result is used by the physician 34 or the medical platform/server 32 to determine, at least in part, proper treatment or proper amount of drug dosage to be administered or prescribed to the user to cure or alleviate the user's movement disorder.

In yet another embodiment of the present disclosure, the disclosed platform 2 includes a cellphone application and doctors' application as discussed below:

Compliant to phone or circuit modes of operation: Users can use the application solely if they wish not to purchase our hardware for initial trials. This feature enables users to benefit from our services via embedded cell phone's sensors for a specified period of time as most people use cellphone's on a daily basis. Precise diagnosis will be provided when using wearable device 16 in coordination with a cellphone application. The present disclosure is capable of detecting both neurological activities of muscles (change in active potentials) as well as biometric characteristics of movement's patterns such as changes in acceleration and limbs' angles. Knowing that there exists 10% of RLS patients who only have muscular activities instead of periodic limb movement (PLM), the hardware will help to diagnose close to 100% of RLS symptoms.

Customized calibration: depending on age, gender and physical, characteristics every person's movement disorder characteristics is different. However, all movement disorder could be detected precisely relative to individual's walking characteristics. In the calibration stage the cell phone application takes a sample data from patients' walking characteristics and further diagnosis movement disorder characteristics relative to the walking characteristics.

Using scientific calculation techniques for diagnosis of movement disorder: the algorithm used by the present disclosure uses vector calculus to detect periodic idle limb motions of movement disorder patients. This method provides both scientific and reliable means for detecting various movement disorders.

Application based registration: every user enters his or her information and doctor's information into the app. This method enables them to easily register into the physician's database.

Graphical representation of limb movement and muscles' neurological changes: this feature enables an easy way of self-diagnosis awareness as it shows the frequency of movement disorder characteristics over a 24-hour period of time.

Locational representation of limb movement and muscles' neurological activities: The geographical spread of movement disorder will enable users and doctors to inspect the effect of the outside home stress on movement disorder symptoms.

Server base communication of real-time data: after the initial diagnosis the processed data will be sent to a server based cloud: thus, enabling the cell phone to be uses as a processing unit and the cloud to be a storage unit. Since the processing and the storage of data are done separately the cellphone's application will consume less power and the cellphone's storage will not be used.

In yet another embodiment of the present disclosure, the platform 2 further comprises a user interface unit. The user interface unit allows the user to adjust algorithm, parameter or calculation used by the artificial intelligent 28 or the diagnosing unit 8 to better match the user's movement characteristics. The user interface may be an independent monitor screen connecting to the platform 2 or be part of the platform 2.

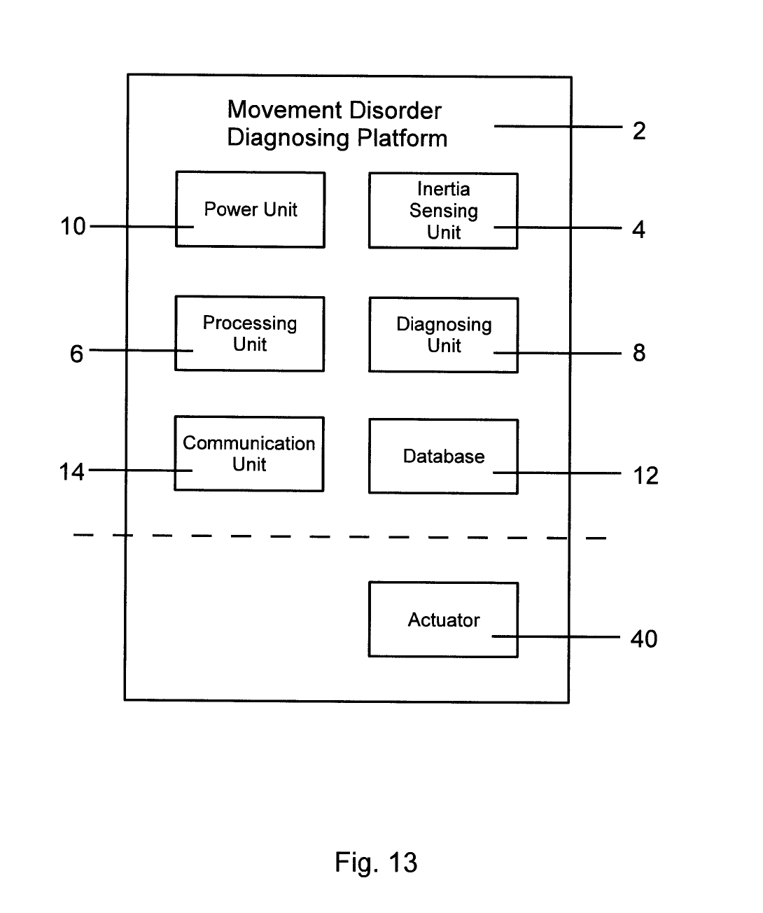

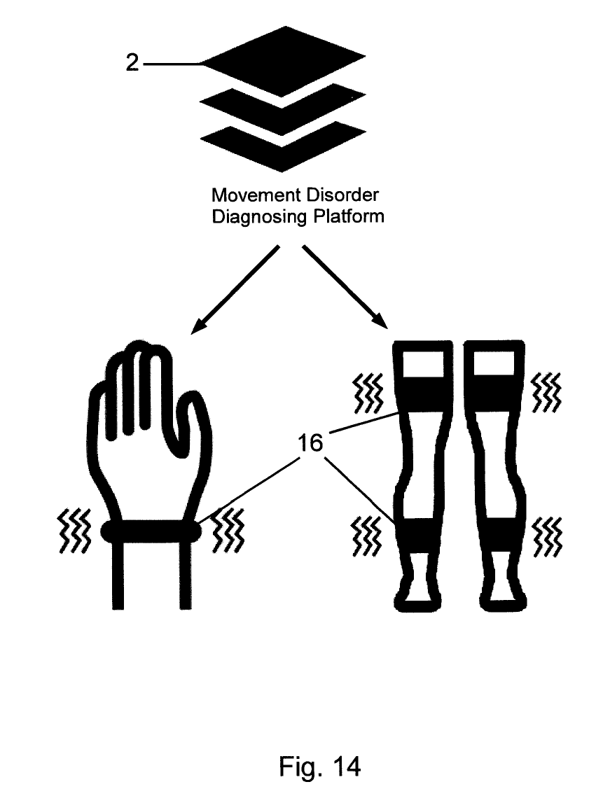

In yet another embodiment of the present disclosure., as shown by FIG. 13, the platform 2 further comprises an actuator 40. Once a movement disorder is detected, the platform 2 alerts the user through the actuator 40 and optionally a cell phone application about the user's unwanted limb movement as shown by FIG. 14.

In yet another embodiment of the present disclosure, the platform 2 further comprises a software interface (e.g., software or mobile application) that allows the user to communicate to a physician or a medical expert with functionalities including but not limited to reporting diagnostic information to the users, or receiving data required for calibrating the diagnosis software.

In yet another embodiment of the present disclosure, a method of detecting movement disorder is disclosed. The method comprises: measuring three-dimensional movement of a user using at least one accelerometer and at least one gyroscope; performing signal processing on the measured three-dimensional movement of the user using fast Fourier Transformation (FFT) or Discrete Fourier Transformation (DFT) to quantify the movement into a signal; comparing the signal to signals in a database, where each of the signals in the database is associated with a normal or an abnormal movement of the user or a typical person; and diagnosing whether the signal measured there-dimensional movement corresponds to a movement disorder based on the comparison. The method may further comprise applying therapeutically effective amount of electrical currents to the user when a movement disorder is diagnosed to alleviate pain or discomfort caused by the movement disorder.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A platform for diagnosing movement disorder, the platform comprising:
at least one inertia-sensing unit that comprises at least one accelerometer, wherein the inertia-sensing unit measures a user's movement;
a power unit that provides power at least to the inertia-sensing unit;
a processing unit that performs signal processing on the user's movement and quantifies the movement into a data;
a diagnosing unit that analyzes the data to diagnose whether a movement disorder occurs by comparing the data to a database, where the database includes at least information about the user's movement characteristic;
a communication unit that establishes a communication with a computer, a mobile device or a computer server.

2. The platform of claim 1, wherein the signal processing performed by the processing unit is Fourier Transformation or its derivatives.

3. The platform of claim 1 further comprises an artificial intelligent unit that uses software algorithm or hardware to learn the frequency of the movement disorder, the periodicity of the movement disorder, or the change in movement characteristics.

4. The platform of claim 1 further comprises a user interface unit, wherein the user can adjust algorithm, parameter or calculation used by the diagnosing unit to better match the user's movement characteristics.

5. The platform of claim 1 further comprises an electromyography (EMG) unit that detects and measures change in active potentials of the user's muscle and diagnoses whether the user's movement is normal, the EMG unit's diagnosis is correlated with the diagnosing unit's diagnosis to enhance the platform's accuracy for diagnosing movement disorder.

6. The platform of claim 5 further comprises a transcutaneous electrical nerve stimulation (TENS) unit, wherein the TENS unit automatically applies electrical currents to the user when an abnormal movement is detected to suppress acute and chronic pain caused by the abnormal movement

7. The platform of claim 1 further comprises an electrocardiogram unit that probes the user's heart activity for determining the heart activity's effect on the movement disorder.

8. The platform of claim 1 further comprises an electroencephalogram unit that probes the user's brain activity for determining the brain activity's effect on the movement disorder.

9. The platform of claim 1, wherein data is categorized into idle periodic movement, non-idle periodic movement, locomotive periodic movement and non-periodic movement by the diagnosing unit.

10. The platform of claim 1, wherein at least the inertia-sensing unit, the power unit and the communication unit are on a device wearable by the user on the user's body part.

11. The platform of claim 1, wherein the diagnosing unit is implemented on one or more devices' microcontrollers, processors or system on chips or be implemented on one or more mobile device electronic devices or be implemented on one or more servers.

12. The platform of claim 1, wherein the communication unit transmits the data directly or indirectly to a physician or a medical platform.

13. A platform for diagnosing movement disorder and alleviating pain caused by the movement disorder, the platform comprising:
at least one inertia-sensing unit that comprises at least one accelerometer, and measures a user's movement;
a power unit that provides power at least to the inertia-sensing unit;
a processing unit that performs signal processing on the user's movement measured and quantifies the movement into a data using Fourier Transformation or a derivative of Fourier Transformation;
a diagnosing unit that analyzes the data to diagnose whether a movement disorder occurs by comparing the data to a database, where the database includes at least information about the user's movement characteristic;
an electromyography unit that detects and measures change in active potentials of the user's muscle; and
a transcutaneous electrical nerve stimulation unit that applies electrical current to the user when a movement disorder is diagnosed to suppress acute and chronic pain caused by the movement order.

14. The platform of claim 13, wherein the transcutaneous electrical nerve stimulation unit has electrodes that are automatically adjustable.

15. The platform of claim 14 further comprises an artificial intelligent unit that uses the electromyography unit to probe whether the electrical current applied to the user is effective and adjusts the electromyography unit's electrical current level accordingly.

16. The platform of claim 15 further comprises a user interface allowing the user to manually adjust the platform's or its components' parameter to assist the artificial intelligent unit in learning or better understanding the user's symptom pattern, and allowing the transcutaneous electrical nerve stimulation unit to apply more accurate amount of electrical current to the user.

17. The platform of claim 13 further comprises an electrocardiogram (EKG or ECG) unit or an electroencephalogram (EEG) unit or both to enhance the diagnosing unit's diagnosing accuracy.

18. The platform of claim 13, wherein the transcutaneous electrical nerve stimulation unit stimulates the user's muscle and looks for the electromyography unit's feedback and the transcutaneous electrical nerve stimulation unit repeats this process until the transcutaneous electrical nerve stimulation unit finds right point of stimulation for the user.

19. A method of detecting movement disorder and alleviating pain caused by the movement disorder comprising:
measuring movement of a user using at least one accelerometer;
performing signal processing on the measured movement of the user to quantify the movement into a data using Fourier Transformation or a derivative of Fourier Transformation;
comparing the data to a database, where the database includes at least information about the user's movement; and
diagnosing whether the data corresponds to a movement disorder based on the comparison.

20. The method of claim 19 further comprises:
applying electrical currents to the user using a transcutaneous electrical nerve stimulation unit when the movement disorder is detected;
measuring change in active potentials of the user's muscle in response to the electrical current using an electromyography unit; and
increasing or decreasing the electrical currents' level based at least in part on the electromyography unit's measurement.
